(19) United States
(12) Patent Application Publication
(10) Pub. No.: US 2026/0166223 A1
(43) Pub. Date: Jun. 18, 2026

(54) POSITIVE DISPLACEMENT DEVICE

(71) Applicant: CareFusion 303, Inc., San Diego, CA (US)

(72) Inventor: Soon Yong PARK, Cypress, CA (US)

(21) Appl. No.: 18/985,667

(22) Filed: Dec. 18, 2024

(51) Int. Cl. A61M 5/168; A61M 5/14; A61M 39/10; A61M 39/28

(52) U.S. Cl. CPC A61M 5/16813; A61M 5/1413; A61M 39/10; A61M 39/287

(57) ABSTRACT

Devices for creating positive fluid displacement are disclosed. Converting a device or system that typically creates a negative fluid displacement, either by design or through accidental misuse of the equipment, into a device or system that creates a positive fluid displacement can minimize risks associated with negative fluid displacement, including risks of infection. Existing devices for use in patient IV lines, such as needle-free connectors and slide clamp designs, can be modified to create positive fluid displacement.

### FIELD OF THE INVENTION

The present disclosure generally relates to fluid displacement in fluid connectors, and in particular, converting negative and neutral displacement needle free connectors to positive displacement connectors.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. In many cases, multiple fluid connector pieces need to be connected to form the complete fluid infusion system. The fluid connector pieces often need to be disconnected and reconnected to each other and to new connector pieces throughout the treatment of a patient.

Certain needleless or needle-free connectors may be used in an IV set and may have a self-sealing port to prevent leakage of fluid when a mating medical implement is decoupled from such a needleless connector. Additionally, a needleless connector may include a mechanical valve, for example, a collapsible valve comprising a flexible material for providing the self-sealing port and controlling the flow of fluid within the IV set. When a needleless connector is used in an IV set and then disconnected, a negative fluid displacement can withdraw blood into a catheter, which is a main cause of catheter occlusion. A majority of current market needle free connectors are negative displacement connectors.

### SUMMARY

One or more embodiments of the present disclosure are directed to a device for creating positive fluid displacement, the device comprising a needle-free connector comprising an outer housing having a first end and a second end, a flexible internal valve at least partially disposed within the housing, and a fluid pathway extending longitudinally between the first end and the second end of the housing, and an opening in the outer housing at which a section of the flexible internal valve is accessible from outside the housing.

In some embodiments, the device has a first end comprising a first fitting with a first port and a second end comprising a second fitting with a second port, where fluid is injected at the first end and flows out of the device at the second end and into IV tubing that connects to a patient.

In some embodiments, the device further comprises a button extending through the opening such that the button contacts and compresses the flexible internal valve when the button is pressed.

In some embodiments, the button is secured to the outer housing by a belt that surrounds the outer housing and permits the button to move through the opening to compress the flexible internal valve and to return to a resting position that does not compress the flexible internal valve.

In some embodiments, a volume of fluid from inside the flexible internal valve is displaced when the button compresses the flexible internal valve, and wherein the volume of displaced fluid flows out of the second end of the device toward the patient.

In some embodiments, the device creates a negative or neutral fluid displacement when the first end of the device is disconnected from a fluid source, and wherein compressing the button to displace the fluid remaining in the flexible valve creates a positive fluid displacement.

One or more embodiments of the present disclosure are directed to a device for creating positive fluid displacement in an intravenous line (IV) where the device comprises a slide clamp comprising a top surface, a first side and a second side at opposing ends of the top surface, and an opening having a widest section at a first end of the opening and a narrow slot extending from the widest section to a second end of the opening with a wall along a perimeter of the opening extending vertically downwards from the top surface of the slide clamp to create a depth of the opening, wherein the widest section permits the IV line to pass through the slide clamp unaffected, and wherein the narrow slot compresses the IV line when the IV line is within the narrow slot.

In some embodiments, the IV line connects a patient to an injection source and is comprised of flexible tubing that is occluded when the IV line is within the narrow slot.

In some embodiments, the device is located on the IV line between a negative displacement needle-free connector and a patient.

In some embodiments, fluid within the IV tubing is unaffected when the slide clamp surrounds the IV tubing at the widest section of the opening and wherein fluid within the IV tubing is displaced from the IV tubing when the slide clamp compresses the IV tubing within the narrow slot.

In some embodiments, the fluid is displaced such that the fluid flows toward the patient, creating a positive fluid displacement.

In some embodiments, the depth of the opening is tapered due to the wall having a greater height at the second end of the opening than at where the narrow slot meets the widest section.

In some embodiments, a longer length of IV tubing is occluded when the IV tubing is at the second end of the opening than when the IV tubing is closer to the widest section of the opening.

One or more embodiments of the present disclosure are directed to a device for creating positive fluid displacement in IV tubing where the device comprises a slide clamp comprising a base, a first side and a second side at opposing ends of the base, and an opening cut through the base and having a widest section at a first end of the opening and a narrow slot extending from the widest section to a second end of the opening, wherein the opening has a vertical depth through the slide clamp, wherein the widest section permits the IV tubing to pass through the slide clamp unaffected, and wherein the narrow slot compresses the IV tubing when the IV tubing is within the narrow slot, and a tubing protector comprising a sleeve extending from a center section, the center section having a channel extending horizontally through the center section, and the sleeve extending above and below the center section perpendicularly to the center section, the sleeve having an inner diameter that permits the IV tubing to extend through the sleeve, and the channel having a width and a height such that the base of the slide clamp can extend through the channel, a first surface attached to and extending along the sleeve, and a second surface attached to and extending along the sleeve on an opposing side of the sleeve from the first surface.

In some embodiments, the sleeve is comprised of a substantially rigid material and is configured to surround a length of IV tubing.

In some embodiments, the slide clamp base is slidable through the channel, and wherein fluid inside the IV tubing is displaced when the IV tubing is compressed in the narrow slot.

In some embodiments, the tubing protector is configured to protect the IV tubing from compressing and causing fluid displacement from touching and compressing the tubing while moving the clamp.

In some embodiments, the slide clamp further comprises a wall extending substantially perpendicularly from one of the first side or the second side of the slide clamp, and wherein the wall provides a surface to apply forces to in order to slide the slide clamp through the channel of the tubing protector.

In some embodiments, the slide clamp further comprises a wall extending substantially perpendicularly from one of the first side or the second side of the slide clamp, and wherein the wall provides a surface to apply forces to in order to slide the slide clamp through the channel of the tubing protector.

In some embodiments, the first surface and the second surface are comprised of a substantially rigid material and are configured to provide a location for a clinician to touch the device to move the slide clamp without touching the IV tubing.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIGS. 1A-1C show an embodiment of a legacy needle-free connector design.

FIGS. 2A-2C show an embodiment of a needle-free connector with a positive displacement design.

FIGS. 3A-3G show an embodiment of a positive displacement device on an IV line.

FIGS. 4A and 4B show an embodiment of a positive displacement device on an IV line.

FIGS. 5A and 5B show an embodiment of a positive displacement device on an IV line.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of fluid connectors, such embodiments can be used in other systems that benefit from creating sterile connections. Furthermore, various applications of such embodiments and modifications thereto, which may occur to those who are skilled in the art, are also encompassed by the general concepts described herein.

Fluid displacement within a needleless connector is typically described as being either negative, positive, or neutral. Connectors with negative displacement allow blood to be pulled back, or to reflux, into the catheter lumen during connection, disconnection, or when an administration set is attached. With some needle-free connectors, a blunt cannula or male luer tip is used to access the connector. However, these can be relatively large, creating negative fluid displacement, or reflux, as they are withdrawn from the needle-free connector. Connectors with positive fluid displacement hold a small reservoir of fluid such that when an IV set or syringe is disconnected, fluid is pushed into the catheter lumen to overcome any blood that refluxed into the lumen. Neutral fluid displacement prevents blood from moving into the catheter lumen upon connection or disconnection.

The disclosed positive fluid displacement devices overcome several challenges discovered with respect to adverse effects associated with negative and neutral fluid displacement. A majority of current market needle-free connectors are negative displacement connectors. Connectors with a negative displacement withdraw fluid into a catheter when they are disconnected from their upstream fluid source. This withdrawal of fluid when the device is disconnected is a main cause of catheter occlusion. The withdrawal of fluid from negative fluid displacement can also increase risks of line infection and central associated bloodstream infections (CLABSI) as well as delayed treatment and overall increases to patient recovery time and hospital stay.

Therefore, in accordance with the present disclosure, it is advantageous to provide devices such as described herein that create positive fluid displacement. Creating positive fluid displacement in an IV line or converting a system that would typically create negative fluid displacement into a system that has a positive fluid displacement can minimize some of the risks associated with negative fluid displacement.

In embodiments of the present disclosure, existing needle-free connector (NFC) designs can be converted into devices that have a positive fluid displacement. Current NFCs may have a flexible valve housed at least partially inside a housing. The flexible valve is deformed when a device is connected to the NFC to provide fluid to a patient. Deforming the valve allows fluid to flow through the NFC and into the patient. However, some NFCs have a negative fluid displacement either by design or when used according to incorrect protocols. Some current NFCs are designed such that when the flexible valve returns to its closed state after disconnection, the internal valve cavity pulls fluid into its expanded cavity, creating negative fluid displacement. Embodiments of the present application can turn NFCs with a negative fluid displacement into NFCs with a positive fluid displacement.

In some embodiments, a button can be added to existing NFC designs that converts the NFC into a positive displacement NFC. A hole may be cut into the side of the housing of the NFC, allowing access to the internal flexible valve from the side of the NFC. The NFC may be connected and disconnected as usual. However, after disconnecting the NFC, a button may be pressed through the hole in the housing of the NFC. The button may comprise a volume of material that makes contact with the flexible valve when the button is depressed through the hole. When the button makes contact with the flexible valve, the valve can be compressed, dispelling any fluid remaining within the valve. By pressing a button on the NFC after the flexible valve returns to its closed state, the internal valve is compressed and the compressed valve pushes the fluid inside the valve cavity into a patient vein. This dispelling of fluid can create a positive fluid displacement.

In some embodiments, the button is connected to the housing of the NFC through a belt that wraps around the exterior of the housing. The belt may be configured to secure the button in place relative to the hole in the housing and to allow the button to be compressed through the hole and to return to its original positioning when the button is not being compressed.

In embodiments of the present disclosure, existing slide clamp designs can be modified to create positive fluid displacement. A positive displacement slide clamp device can be added onto an IV tube below a negative displacement NFC. After disconnecting the NFC, the IV tube can be slid into a narrow slot on the positive displacement device by a nurse. Then, the positive displacement device compresses the tube connected to a catheter and displaces fluid in the tube into a patient. This can reduce catheter occlusion. The narrow section of the slide clamp, which pinches the IV tubing closed when the clamp is slid to place the tubing within the narrow section, may have an increased depth in order to displace a greater volume of fluid.

In some embodiments, the positive displacement slide clamp has a depth that gradually increases from the area that can surround the IV tubing without pinching the IV tubing to the end of the narrow section of the slide clamp that pinches the IV tubing. This gradual increase in depth can assist in more gently pinching the tubing, starting with a smaller pinching depth, and can direct the displaced fluid as the depth increases as the tubing is moved through the narrow section of the clamp and the depth increases, pinching more IV tubing and thus displacing more fluid.

In some embodiments, the positive displacement slide clamp has a tube clamping rib. The clamping rib begins at the edge of the wide portion of the through hole in the slide clamp. When IV tubing is pushed to the middle area of the positive displacement slide clamp, the tube is occluded by the tube clamping rib. Further insertion of tubing to the back of the clamp squeezes the tubing and displaces fluid in the tube into the patient, creating positive fluid displacement. In some embodiments, the tube clamping rib is thinnest at the beginning of the narrow section of the slide clamp and has an increasing depth with a widest depth at the end of the narrow section.

In some embodiments, an alternate positive displacement device is a pinch clamp mechanism with a widened pinching area to achieve desired displacement volume. When the pinch clamp mechanism with a widened pinching area is used, fluid displacement due to the widened pinching area can lead to positive fluid displacement. Use of this positive displacement device after the disconnection of a fluid connector without a positive fluid displacement can instead create positive fluid displacement, mitigating risks associated with negative fluid displacement.

In some embodiments, a positive displacement slide clamp can be used in conjunction with a tubing protector. Current slide clamps in the market are designed to be a neutral displacement clamp, which neither withdraws blood into a catheter nor injects IV fluid into a patient while occluding an IV line. However, a majority of slide clamps are found to be negative displacement devices, which withdraw blood into a catheter after clamping an IV line because a nurse pinches and releases the patient side of the IV line to activate the slide clamp. The negative displacement created by slide clamp use from the pinching and releasing of the IV line withdraws blood or fluid into a catheter. This withdrawal of fluid from negative fluid displacement is a main cause of catheter occlusion related infections such as peripheral line-associated bloodstream infection (PLABSI).

In some embodiments of the present disclosure, the negative displacement problem with current slide clamp designs can be solved by introducing a neutral displacement tubing protector, which has sleeves for IV tubing just above and below a slide clamp. The tubing protector provides a place to pull tubing to activate the slide clamp and eliminate tubing pinching which creates negative fluid displacement.

In some embodiments, the tubing protector comprises a central section that has a slot open through the center such that the slide clamp can pass through the slot. The central section may have a top face and a bottom face that are parallel with the slide clamp. Sleeve sections may extend from both the top face and the bottom face of the tubing protector. The sleeve sections may be proportioned such that IV tubing can extend through the sleeve sections. The sleeve sections of the tubing protector and the slide clamp may also be positioned such that IV tubing can be encased within the sleeve sections and pass through the slot of the slide clamp simultaneously. The slide clamp may move through the slot of the central section of the tubing protector, moving the IV tubing within the different clamping sections of the slide clamp, while the IV tubing remains encased within the sleeve sections. The sleeve sections may surround the IV tubing with enough open space that the IV tubing can be deformed when it is pinched within the narrow section of the slide clamp and still fit within the sleeve sections. The sleeve sections may be made from a stiff or substantially stiff material such that the IV tubing is protected from being compressed, bent, or otherwise misshapen while protected within the sleeve sections because the sleeve sections cannot be easily misshapen.

In some embodiments, the tubing protector comprises wall portions extending from the sleeve sections. The wall portions may provide areas to hold or push the tubing protector relative to the slide clamp. The wall portions may also provide additional support to the structure of the tubing protector. The tubing protector may comprise two wall portions that extend on opposite sides of the sleeve sections aligned with the longitudinal edges of the slide clamp. The wall portions may extend around the slot in the central section of the tubing protector, connecting both sleeve sections.

In some embodiments, the slide clamp that is used with the tubing protector may have a wall on one or more ends of the slide clamp that extends vertically from a longitudinal end of the slide clamp. This wall can assist in moving the slide clamp relative to the tubing protector by providing a surface to press on. Additionally, in some embodiments, the wall may have a curved shape so that the wall cannot be fully aligned with a wall portion of the tubing protector. This curved shape may ensure that even when the slide clamp is pushed through the tubing protector to place the IV line at a far end of the slide clamp slot, there will be a gap between the wall of the slide clamp and the wall portions attached to the sleeve portions of the tubing protector. This may ensure that the slide clamp wall can be used to pull the slide clamp through the tubing protector.

In some embodiments, the slide clamp and the tubing protector are both made from stiff or substantially stiff materials, such as plastic or metal, to allow for nurses, doctors, clinicians, or other people or things to interact with the slide clamp and the tubing protector without deforming the slide clamp or the tubing protector, and thus without accidentally deforming the IV tubing when moving the slide clamp to clamp or unclamp the IV tubing. This protection from pressing on and releasing the IV tubing while using the slide clamp can minimize accidental negative fluid displacement from touching the IV line, and thus compressing the IV tubing, when moving the slide clamp and then releasing the tubing. The tubing protector provides a place for fingers to activate the slide clamp and eliminates tubing pinching that creases negative fluid displacement.

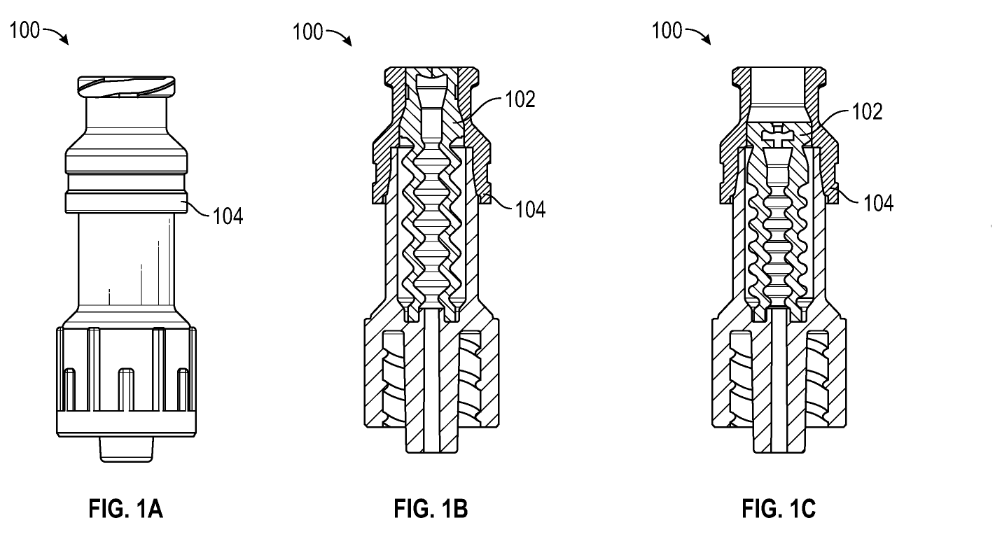

FIG. 1A shows an exemplary legacy needle-free connector (NFC). The legacy NFC 100 has a housing 104. The NFC 100 has a first end and second end, and fluid is able to flow through the device. A needle-free connection point at the first end allows male luers and other blunt fluid injection devices to access a flexible valve area. The flexible valve can deform to allow a connector to connect to the NFC and form a fluid pathway through the NFC. Injected fluid can flow through or around the flexible valve to exit the device at the second end and continue flowing through IV tubing or other fluid pathways connected to the second end of the device. The housing 104 provides a rigid or semi-rigid material to protect and cover the flexible internal valve and to provide structure for threads, lips, or other design features to allow the NFC 100 to be connected to fluid lines, injection sources, and other fluid connectors.

FIG. 1B shows the exemplary NFC 100 in a cross-sectional view. The legacy NFC design 100 has a flexible valve 102 and a housing 104. The flexible valve 102 is shown in a disconnected or closed state. The NFC 100 has a first end and a second end, and fluid is able to flow through the device. A split-septum design at the first end allows blunt injection luers to connect with the flexible internal valve 102. A threaded second end allows the NFC 100 to be connected to other threaded connectors to allow for connection to connect to a patient’s IV line.

FIG. 1C shows the exemplary NFC 100 in a cross-sectional view with the flexible valve 102 shown in a connected or open state. When the flexible valve 102 is compressed at the first end of the NFC 100 by a blunt connection luer, the valve compressed into the housing, allowing fluid to pass through the NFC 100.

Many NFCs such as shown in FIGS. 1A-1C create a negative fluid displacement when the flexible valve returns to its closed state. The internal valve cavity may pull fluid back into the cavity when the valve returns to its expanded or closed state.

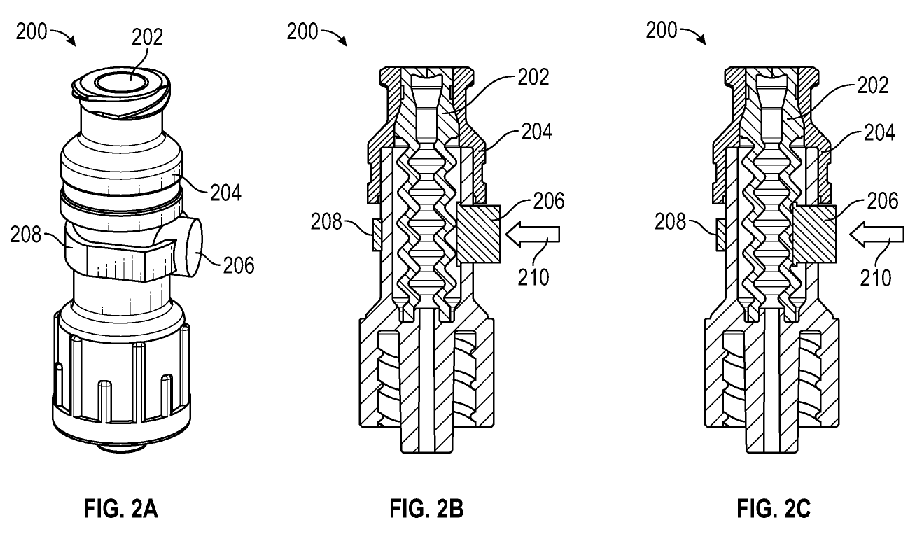

FIG. 2A shows an embodiment of an NFC with a positive displacement design. The NFC 200 has a flexible valve 202 at least partially inside a housing 204. A button 206 is secured by a belt 208 to the housing 204. The NFC 200 modifies existing NFC designs to convert designs that have a negative fluid displacement into a design that creates a positive fluid displacement.

FIG. 2B shows a cross-sectional view of an embodiment of an NFC with a positive displacement attachment not in use. The NFC 200 has a flexible valve 202 at least partially inside a housing 204. A button 206 is secured by a belt 208 to the housing 204. A force 210 may be used to press on the button 206.

FIG. 2C shows a cross-sectional view of an embodiment of an NFC with a positive displacement attachment in use. The NFC 200 has a flexible valve 202 at least partially inside a housing 204. A button 206 is secured by a belt 208 to the housing 204. A force 210 may be used to press on the button 206. FIG. 2C shows the button 206 compressed by the force 210 such that the button 206 contacts the flexible valve 202. By pressing in on the flexible valve 202, the button 206 can displace fluid from inside the flexible valve 202. The button 206 as shown has a largely cylindrical shape, although it should be understood that the button may have a variety of different shapes, including rectangular prism or other regular or irregular shapes capable of extending through a hole and providing a surface to press on and surface to make contact with the flexible valve 202. The button 206 as shown has an internal lip that protrudes outward from a surface of the button 206 that extends within the housing 204. This lip provides structure to prevent the button 206 from falling out of its place within the hole in the housing 204 in which it resides, as the lip extends beyond the dimensions of the hole. The button 206 is shown with a lip that extends around a portion of the button that remains within an internal cavity of the housing, however, it should be understood that embodiments of the button 206 may have a lip that extends in a different fashion or may not have a lip portion at all.

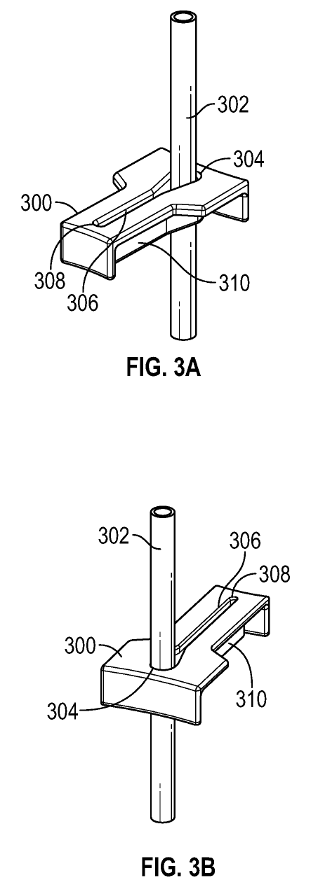

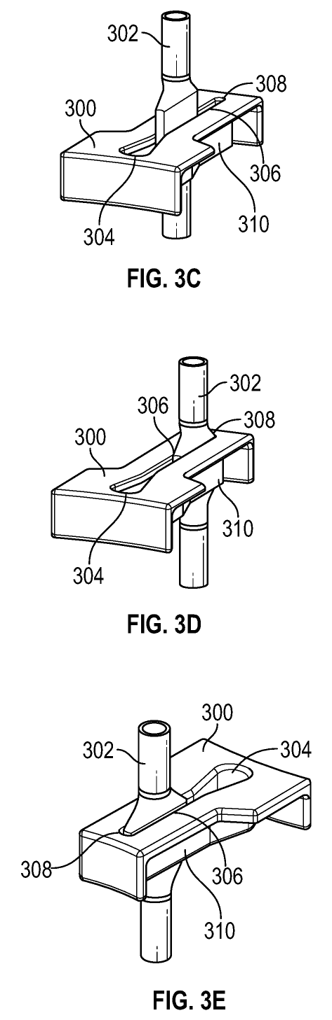

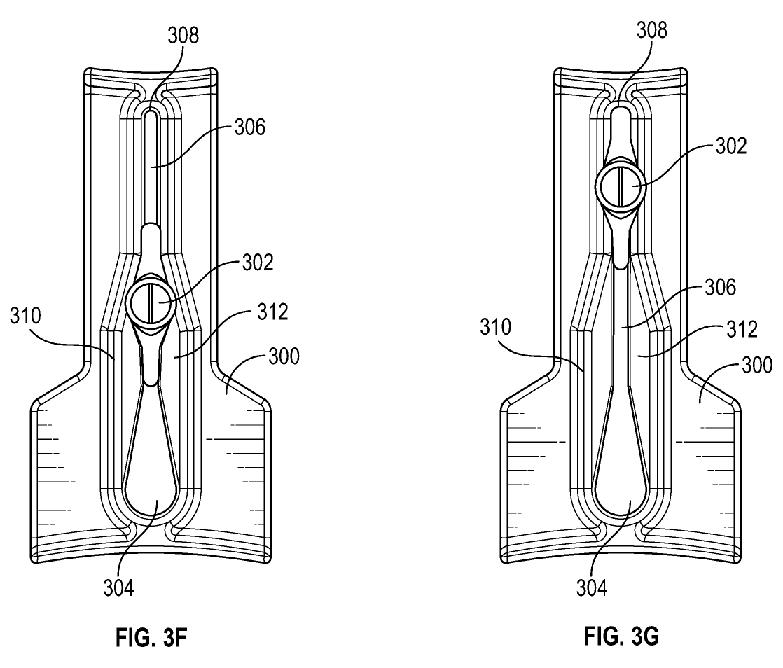

FIG. 3A shows an embodiment of a positive displacement device on an IV line, with the tubing of the IV line uncompressed. The positive displacement device 300 is a modified slide clamp on an IV line 302. The slide clamp has a slot with a widest section 304 and a narrow slot 306 that connects from the widest section 304 to a slot end 308. The positive displacement device 300 has a depth created by a wall 310. The IV line 302 passes through the positive displacement device 300 and is unaffected when the IV line 302 passes through the device at the widest section 304. FIG. 3B shows a second view of the positive displacement device on an IV line of FIG. 3A.

As depicted in FIGS. 3A and 3B, the IV line 302 can pass through the positive displacement device 300 at the widest section 304 of the slide clamp slot without affecting the IV line 302. The widest section 304 may have a teardrop shape such as shown, with the widest section 304 having a diameter that tapers into the narrow slot 306. However, it should also be understood that the widest section may have other shapes that permit the IV line 302 to pass through the shape without being pinched by the slide clamp slot. The diameter of the widest section 304 is as least as large as the outer diameter of the IV line 302.

FIG. 3C shows an embodiment of a positive displacement device on an IV line, with the tubing of the IV line compressed. When the slide clamp is slid so that the clamp squeezes the IV line 302 within the narrow slot 306, the IV line 302 is compressed. FIG. 3D shows an embodiment of a positive displacement device on an IV line, with the tubing of the IV line compressed. The IV line 302 is maximally compressed when the IV line 302 reaches the slot end 308. FIG. 3E shows a second view of the positive displacement device on an IV line of FIG. 3D.

FIG. 3F shows a bottom view of the positive displacement device on an IV line of FIG. 3C. FIG. 3G shows a bottom view of the positive displacement device on an IV line of FIGS. 3D and 3E. The IV line 302 is compressed within the narrow slot 306 when it is located between the widest section 304 and the slot end 308. The wall 310 extends around the slot, and clamping ribs 312 extend from the wall 310 to form the shape of the slide clamp slot. The clamping ribs 312 provide a thin area to first clamp the IV line 302. The clamping ribs 312 occlude the IV line 302. Further insertion of the IV line 302 to the slot end 308 squeezes the tubing of the IV line 302 and displaces fluid into the tubing into the patient, creating a positive fluid displacement. In some embodiments, the clamping ribs 312 are the thinnest at their first protrusion near the widest section 304 of the slide clamp slot and have an increasing depth until the far end of the slide clamp slot at the slot end 308. The increasing depth can direct the fluid displacement, and an increased depth displaces an increased amount of fluid, leading to larger amounts of fluid in the positive fluid displacement.

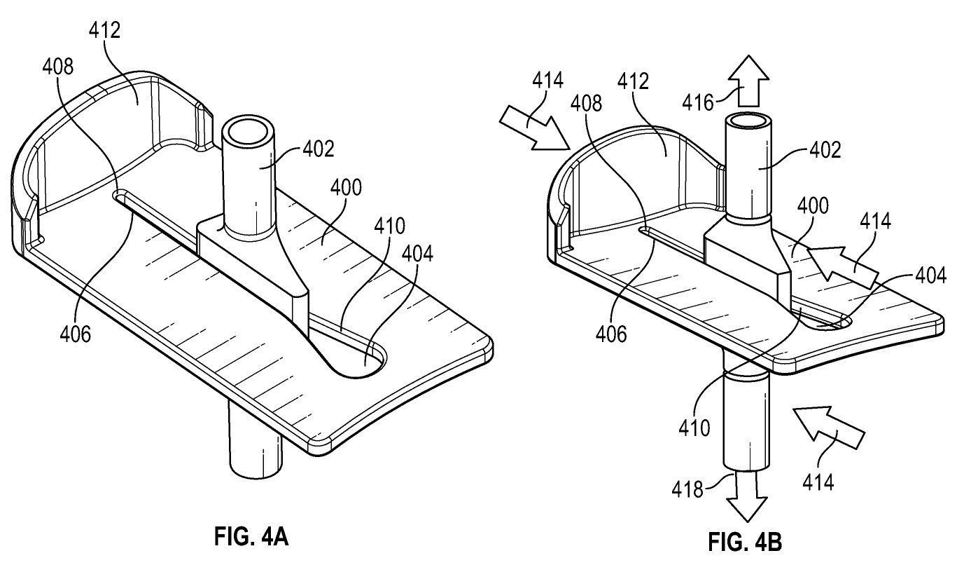

FIG. 4A shows an embodiment of a slide clamp design. The slide clamp 400 is positioned along an IV line 402. The slide clamp 400 has an opening with a widest section 404 and a narrow slot 406 that extends from the widest section 404 to a slot end 408. The opening has a depth 410. When the IV line 402 is pushed into the narrow slot 406 of the slide clamp 400, the IV line 402 compresses. The slide clamp 400 additionally comprises a wall 412 that extends perpendicularly or substantially perpendicularly from the main portion of the slide clamp with the slide clamp slot. The wall 412 may provide a location to apply force to the slide clamp 400 to move the slide clamp relative to the IV line 402 in order to clamp or unclamp the IV line 402. The wall 412 may extend in one or both directions (upwards and/or downwards) from the main portion of the slide clamp with the slide clamp slot. The wall 412 may extend from the side of the slide clamp nearest to the slot end 408 or from the side nearest to the widest section 404 or from both opposing ends. As shown, the wall 412 may have curved edges to better interact with other related equipment or provide an indication of where to apply force when using the device. However, it should also be understood that the wall 412 may have a variety of similar orientations and shapes.

FIG. 4B shows an embodiment of a slide clamp design as shown in FIG. 4A with application forces shown. Forces 414 applied to the IV line 402 and the wall 412 move the IV line 402 within the opening of the slide clamp 400. When the IV line 402 is pushed into the narrow slot 406 of the slide clamp 400, the IV line 402 compresses. Fluid within the IV line 402 when the line is compressed is pushed out of the IV line in the direction 416 and/or in the direction 418.

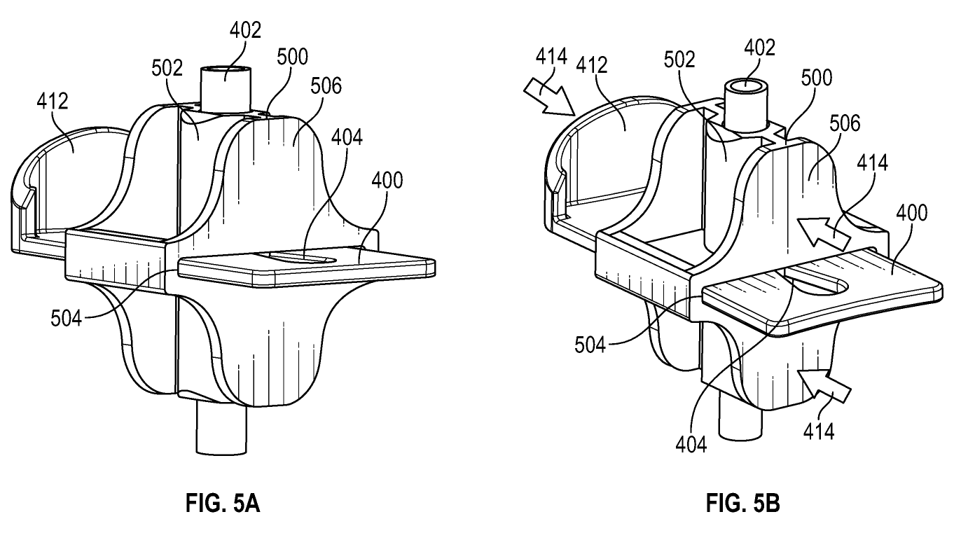

FIG. 5A shows an embodiment of a slide clamp design with a tubing protector. The slide clamp 400 of FIG. 4A and FIG. 4B is shown with the additional element of a tubing protector 500. The tubing protector 500 has a sleeve section 502 and an opening 504. The opening 504 is configured to allow the slide clamp 400 to slide through the opening. The opening 504 can surround a section of the slide clamp 400. The opening 504 has a top face and a bottom face, and the sleeve section 502 protrudes from both the top face and the bottom face. The sleeve section 502 is configured to surround the IV line 402, providing a protective barrier to the section of IV tubing housed within the sleeve section 502. The sleeve section 502 aligns with the slide clamp 400 such that the IV line 402 can extend through the sleeve section 502 and the clamping slot of the slide clamp such that the slide clamp 400 can be moved relative to the tubing protector 500 to place the IV line 402 in the widest section 404 of the slide clamp or the narrow slot 406 of the slide clamp. The sleeve section 502 can surround the IV line 402 when the line is uncompressed within the widest section 404 and when the IV line 402 is pinched closed in the narrow slot 406.

In some embodiments, one or more wall portions 506 can extend from the sleeve sections 502. The wall portions 506 can extend outwards from the sleeve section 502 and create a flat surface that extends perpendicularly to the slide clamp. The wall portions 506 may extend above and below the opening 504. The wall portions 506 may also extend from two coaxial locations on the sleeve section 502 such that the wall portions 506 extend perpendicularly to a central axis of the clamping slot of the slide clamp. In some embodiments, the wall portions 506 may extend substantially parallel to a wall 412 of the slide clamp.

In some aspects of the disclosure, the tubing protector 500 may be used with positive displacement device 300. Here, the opening 504 is configured to allow the positive displacement device 300 to slide through the opening 504, which can then surround a section of the positive displacement device 300. The tubing protector 500 may be used with IV line 302 and positive displacement device 300 in the same manner as discussed above regarding tubing protector 500 being used with IV line 402 and slide clamp 400.

FIG. 5B shows an embodiment of a slide clamp design with a tubing protector with application forces 414 shown. As depicted, forced can be applied to the slide clamp 400 and to the tubing protector 500. The wall 412 of the slide clamp 400 and the wall portions 506 of the tubing protector provide surfaces upon which to apply these forces 414. The forces 414 can also be reversed or applied to different locations on the slide clamp 400 and the tubing protector 500 to move the slide clamp 400 and the tubing protector 500 relative to each other in an opposite direction. By providing surfaces to apply forces to rather than applying forces directly to the IV line, the IV line above and below the slide clamp is protected from being compressed and uncompressed due to being touched while the slide clamp is moved by a nurse or other user. By removing inadvertent squeezing of the IV tubing, the tubing protector 500 minimizes instances of creating inadvertent negative fluid displacement. Minimizing negative fluid displacement minimizes risk of infection due to negative fluid displacement within the IV line to the patient.

The disclosures described herein include at least the following clauses:

Clause 1: A device for creating positive fluid displacement through a needle-free connector, the device comprising: the needle-free connector, comprising: an outer housing having a first end and a second end; a flexible internal valve at least partially disposed within the housing; and a fluid pathway extending longitudinally between the first end and the second end of the housing; and an opening in the outer housing at which a section of the flexible internal valve is accessible from outside the housing.

Clause 2: The device of Clause 1, wherein the device has a first end comprising a first fitting with a first port and a second end comprising a second fitting with a second port, where fluid is injected at the first end and flows out of the device at the second end and into IV tubing that connects to a patient.

Clause 3: The device of Clause 2, further comprising a button extending through the opening such that the button contacts and compresses the flexible internal valve when the button is pressed.

Clause 4: The device of Clause 3, wherein the button is secured to the outer housing by a belt that surrounds the outer housing and permits the button to move through the opening to compress the flexible internal valve and to return to a resting position that does not compress the flexible internal valve.

Clause 5: The device of Clause 3, wherein a volume of fluid from inside the flexible internal valve is displaced when the button compresses the flexible internal valve, and wherein the volume of displaced fluid flows out of the second end of the device toward the patient.

Clause 6: The device of Clause 5, wherein the device creates a negative or neutral fluid displacement when the first end of the device is disconnected from a fluid source, and wherein compressing the button to displace the fluid remaining in the flexible valve creates a positive fluid displacement.

Clause 7: A device for creating positive fluid displacement in an intravenous line (IV), the device comprising: a slide clamp comprising: a top surface; a first side and a second side at opposing ends of the top surface; and an opening having a widest section at a first end of the opening and a narrow slot extending from the widest section to a second end of the opening with a wall along a perimeter of the opening extending vertically downwards from the top surface of the slide clamp to create a depth of the opening, wherein the widest section permits the IV line to pass through the slide clamp unaffected, and wherein the narrow slot compresses the IV line when the IV line is within the narrow slot.

Clause 8: The device of Clause 7, wherein the IV line connects a patient to an injection source and is comprised of flexible tubing that is occluded when the IV line is within the narrow slot.

Clause 9: The device of Clause 8, wherein the device is located on the IV line between a needle-free connector and a patient.

Clause 10: The device of Clause 8, wherein fluid within the IV tubing is unaffected when the slide clamp surrounds the IV tubing at the widest section of the opening, wherein the IV tubing is occluded when the IV tubing is disposed in a first portion of the narrow slot and wherein fluid within the IV tubing is displaced from the IV tubing when the slide clamp compresses the IV tubing within a second portion of the narrow slot.

Clause 11: The device of Clause 10, wherein the fluid is displaced such that the fluid flows toward the patient, creating a positive fluid displacement.

Clause 12: The device of Clause 8, wherein the depth of the opening is tapered due to the wall having a greater height at the second end of the opening than at where the narrow slot meets the widest section.

Clause 13: The device of Clause 12, wherein a longer length of IV tubing is occluded when the IV tubing is at the second end of the opening than when the IV tubing is closer to the widest section of the opening.

Clause 14: A device for creating positive fluid displacement in IV tubing, the device comprising: a slide clamp comprising: a base; a first side and a second side at opposing ends of the base; and an opening cut through the base and having a widest section at a first end of the opening and a narrow slot extending from the widest section to a second end of the opening; wherein the opening has a vertical depth through the slide clamp, wherein the widest section permits the IV tubing to pass through the slide clamp unaffected, and wherein the narrow slot compresses the IV tubing when the IV tubing is within the narrow slot, and a tubing protector comprising: a sleeve extending from a center section, the center section having a channel extending horizontally through the center section, and the sleeve extending above and below the center section perpendicularly to the center section, the sleeve having an inner diameter that permits the IV tubing to extend through the sleeve, and the channel having a width and a height such that the base of the slide clamp can extend through the channel; a first surface attached to and extending along the sleeve; and a second surface attached to and extending along the sleeve on an opposing side of the sleeve from the first surface.

Clause 15: The device of Clause 14, wherein the sleeve is comprised of a substantially rigid material and is configured to surround a length of IV tubing.

Clause 16: The device of Clause 14, wherein the slide clamp base is slidable through the channel, and wherein fluid inside the IV tubing is displaced when the IV tubing is compressed in the narrow slot.

Clause 17: The device of Clause 16, wherein the tubing protector is configured to protect the IV tubing from compressing and causing fluid displacement from touching and compressing the tubing while moving the clamp.

Clause 18: The device of Clause 14, wherein the slide clamp further comprises a wall extending substantially perpendicularly from one of the first side or the second side of the slide clamp, and wherein the wall provides a surface to apply forces to in order to slide the slide clamp through the channel of the tubing protector.

Clause 19: The device of Clause 18, wherein the wall of the slide clamp extends upwards from one of the first side or the second side of the slide clamp and curves inward toward the slide clamp opening at one or more ends of the wall.

Clause 20: The device of Clause 14, wherein the first surface and the second surface are comprised of a substantially rigid material and are configured to provide a location for a clinician to touch the device to move the slide clamp without touching the IV tubing.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. A device for creating positive fluid displacement, the device comprising:
a needle-free connector, comprising:
an outer housing having a first end and a second end;
a flexible internal valve at least partially disposed within the housing; and
a fluid pathway extending longitudinally between the first end and the second end of the housing; and
an opening in the outer housing at which a section of the flexible internal valve is accessible from outside the housing.

2. The device of claim 1, wherein the device has a first end comprising a first fitting with a first port and a second end comprising a second fitting with a second port, where fluid is injected at the first end and flows out of the device at the second end and into IV tubing that connects to a patient.

3. The device of claim 2, further comprising a button extending through the opening such that the button contacts and compresses the flexible internal valve when the button is pressed.

4. The device of claim 3, wherein the button is secured to the outer housing by a belt that surrounds the outer housing and permits the button to move through the opening to compress the flexible internal valve and to return to a resting position that does not compress the flexible internal valve.

5. The device of claim 3, wherein a volume of fluid from inside the flexible internal valve is displaced when the button compresses the flexible internal valve, and wherein the volume of displaced fluid flows out of the second end of the device toward the patient.

6. The device of claim 5, wherein the device creates a negative or neutral fluid displacement when the first end of the device is disconnected from a fluid source, and wherein compressing the button to displace the fluid remaining in the flexible valve creates a positive fluid displacement.

7. A device for creating positive fluid displacement in an intravenous line (IV), the device comprising:
a slide clamp comprising:
a top surface;
a first side and a second side at opposing ends of the top surface; and
an opening having a widest section at a first end of the opening and a narrow slot extending from the widest section to a second end of the opening with a wall along a perimeter of the opening extending vertically downwards from the top surface of the slide clamp to create a depth of the opening,
wherein the widest section permits the IV line to pass through the slide clamp unaffected, and
wherein the narrow slot compresses the IV line when the IV line is within the narrow slot.

8. The device of claim 7, wherein the IV line connects a patient to an injection source and is comprised of flexible tubing that is occluded when the IV line is within the narrow slot.

9. The device of claim 8, wherein the device is located on the IV line between a needle-free connector and a patient.

10. The device of claim 8, wherein fluid within the IV tubing is unaffected when the slide clamp surrounds the IV tubing at the widest section of the opening, wherein the IV tubing is occluded when the IV tubing is disposed in a first portion of the narrow slot and wherein fluid within the IV tubing is displaced from the IV tubing when the slide clamp compresses the IV tubing within a second portion of the narrow slot.

11. The device of claim 10, wherein the fluid is displaced such that the fluid flows toward the patient, creating a positive fluid displacement.

12. The device of claim 8, wherein the depth of the opening is tapered due to the wall having a greater height at the second end of the opening than at where the narrow slot meets the widest section.

13. The device of claim 12, wherein a longer length of IV tubing is occluded when the IV tubing is at the second end of the opening than when the IV tubing is closer to the widest section of the opening.

14. A device for creating positive fluid displacement in IV tubing, the device comprising:
a slide clamp comprising:
a base;
a first side and a second side at opposing ends of the base; and
an opening cut through the base and having a widest section at a first end of the opening and a narrow slot extending from the widest section to a second end of the opening;
wherein the opening has a vertical depth through the slide clamp,
wherein the widest section permits the IV tubing to pass through the slide clamp unaffected, and
wherein the narrow slot compresses the IV tubing when the IV tubing is within the narrow slot, and
a tubing protector comprising:
a sleeve extending from a center section, the center section having a channel extending horizontally through the center section, and the sleeve extending above and below the center section perpendicularly to the center section, the sleeve having an inner diameter that permits the IV tubing to extend through the sleeve, and the channel having a width and a height such that the base of the slide clamp can extend through the channel;
a first surface attached to and extending along the sleeve; and
a second surface attached to and extending along the sleeve on an opposing side of the sleeve from the first surface.

15. The device of claim 14, wherein the sleeve is comprised of a substantially rigid material and is configured to surround a length of IV tubing.

16. The device of claim 14, wherein the slide clamp base is slidable through the channel, and wherein fluid inside the IV tubing is displaced when the IV tubing is compressed in the narrow slot.

17. The device of claim 16, wherein the tubing protector is configured to protect the IV tubing from compressing and causing fluid displacement from touching and compressing the tubing while moving the clamp.

18. The device of claim 14, wherein the slide clamp further comprises a wall extending substantially perpendicularly from one of the first side or the second side of the slide clamp, and wherein the wall provides a surface to apply forces to in order to slide the slide clamp through the channel of the tubing protector.

19. The device of claim 18, wherein the wall of the slide clamp extends upwards from one of the first side or the second side of the slide clamp and curves inward toward the slide clamp opening at one or more ends of the wall.

20. The device of claim 14, wherein the first surface and the second surface are comprised of a substantially rigid material and are configured to provide a location for a clinician to touch the device to move the slide clamp without touching the IV tubing.
